(19) United States
(12) Patent Application Publication
(10) Pub. No.: US 2026/0283578 A1
(43) Pub. Date: Sep. 24, 2026

(54) BRAIN IMAGING SYSTEM AND BRAIN IMAGING METHOD

(71) Applicant: A-moy Limited, Apia (WS)

(72) Inventor: FAN-PEI YANG, Hsinchu City (TW)

(21) Appl. No.: 19/574,498

(22) Filed: Mar. 23, 2026

Related U.S. Application Data
(63) Continuation-in-part of application No. 18/123,410, filed on Mar. 20, 2023, now abandoned.
Application No. 18/123,410 is a continuation-in-part of application No. 16/366,431, filed on Mar. 27, 2019, now abandoned.

(30) Foreign Application Priority Data
Mar. 30, 2018 (TW) 107111316

(51) Int. Cl. A61B 6/00; A61B 6/03; A61B 6/50; G06T 7/00; G06V 10/774; G06V 10/776; G06V 10/82

(52) U.S. Cl. CPC A61B 6/5217; A61B 6/03; A61B 6/481; A61B 6/501; A61B 6/5235; G06T 7/0012; G06V 10/774; G06V 10/776; G06V 10/82; G06T 2207/10081; G06T 2207/10088; G06T 2207/20084; G06T 2207/30016; G06T 2207/30096; G06T 2207/30104; G06V 2201/031

(57) ABSTRACT

A brain imaging system includes a first imaging device, a second imaging device and a processor. The first imaging device captures first brain images by scanning a patient, and the second imaging device captures second brain images. The processor is configured to: receive and distinguish the first and second brain images; convert the first and second brain images to a first format and a second format; identify and analyzing first features and second features in the converted first and second brain images to obtain cerebral perfusion data and brain lesion data by a first deep learning model and a second deep learning model. A brain imaging method is operated with the brain imaging system.

### CROSS-REFERENCE TO RELATED PATENT APPLICATION

This application is a continuation-in-part application of the U.S. application Ser. No. 18/123,410, filed on Mar. 20, 2023 and entitled “BRAIN IMAGING SYSTEM AND BRAIN IMAGING METHOD”, now pending, the entire disclosures of which are incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an imaging system and an imaging method, and more particularly to a brain imaging system and a brain imaging method.

### BACKGROUND OF THE DISCLOSURE

Nuclear magnetic resonance (NMR) is a non-invasive way to detect human bodies. It obtains variations of magnetic dipole moment of water molecules through transmitting and receiving radio frequency signals, and further to differentiate normal and lesion tissues by using contrast agents. The computerized tomography (CT) is used to obtain a two-dimensional image by having X-rays scanned through a human body. However, the two-dimensional image can only be interpreted by medical staff, which may adversely affect the precision and efficiency thereof.

### SUMMARY OF THE DISCLOSURE

In response to the above-referenced technical inadequacies, the present disclosure provides a brain imaging system and a brain imaging method.

In order to solve the above-mentioned problems, one of the technical aspects adopted by the present disclosure is to provide a brain imaging system, which includes a first imaging device, a second imaging device and a processor. The first imaging device is configured to capture a plurality of first brain images that provide cerebral data representing a contrast agent in a brain of the patient over time by scanning a patient. The second imaging device is configured to capture a plurality of second brain images by scanning the patient. The processor is electrically connected to the first imaging device and the second imaging device, and the processor is configured to: receive the first brain images from the first imaging device and the second brain images from the second imaging device, and distinguish the first brain images and the second brain images; convert the first brain images into a first format; identify first features in the converted first brain images by a first deep learning model; obtain cerebral perfusion data by analyzing the first features identified in the converted first brain images through the first deep learning model; convert the second brain images into a second format; identify second features in the converted second brain images by a second deep learning model; and obtain brain lesion identification data by analyzing the second features identified in the converted second brain images through the second deep learning model. The first deep learning model includes a long-short-term memory (LSTM) neural network, by which the first features in the converted first brain images are identified. The second deep learning model includes you-look-only-once algorithm (YOLO) and region-convolutional neural network (R-CNN), by both of which the second features in the converted second images are identified.

In order to solve the above-mentioned problems, another one of the technical aspects adopted by the present disclosure is to provide a brain imaging method, including: configuring a first imaging device to capture a plurality of first brain images that provide cerebral data representing a contrast agent by scanning a patient; configuring a second imaging device to capture a plurality of second brain images by scanning a patient; and configuring a processor, which is electrically connected to the first imaging device and the second imaging device, to: receive the first brain images from the first imaging device and the second brain images from the second imaging device, and distinguish the first brain images and the second brain images; convert the first brain images into a first format; identify first features in the converted first brain images by a first deep learning model; obtain cerebral perfusion data by analyzing the first features identified in the converted first brain images through the first deep learning model; convert the second brain images into a second format; identify second features in the converted second brain images by a second deep learning model; and obtain brain lesion identification data by analyzing the second features identified in the converted second brain images through the second deep learning model. The first deep learning model includes a long-short-term memory (LSTM) neural network, by which the first features in the converted first brain images are identified. The second deep learning model includes you-look-only-once algorithm (YOLO) and region-convolutional neural network (R-CNN), by both of which the second features in the converted second images are identified.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 shows a block diagram of a brain imaging system according to one embodiment of the present disclosure;

FIG. 2 is a schematic diagram showing a flow path of a contrast agent according to one embodiment of the present disclosure;

FIG. 3 shows a flowchart of a brain imaging method according to one embodiment of the present disclosure;

FIG. 4 shows a detailed flowchart of training the first deep learning model mentioned in FIG. 3;

FIG. 5 shows a detailed flowchart of training the second deep learning model mentioned in FIG. 3;

FIG. 6 is a curve diagram showing an accumulated concentration function of a contrast agent according to one embodiment of the present disclosure;

FIG. 7 is a curve diagram showing a residual concentration function of a contrast agent according to one embodiment of the present disclosure;

FIG. 8 shows a schematic diagram of a lesion map according to one embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

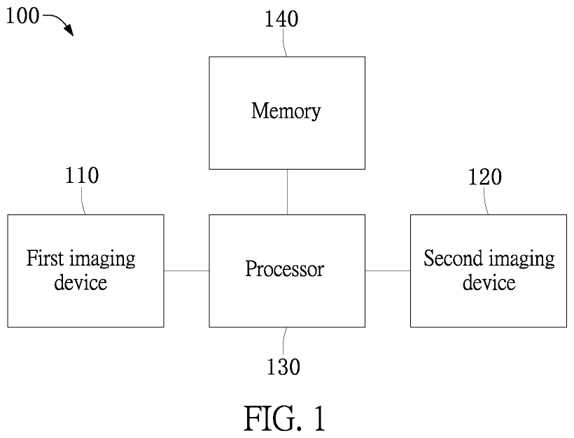

FIG. 1 shows a block diagram of a brain imaging system according to one embodiment of the present disclosure. Reference is made to FIG. 1, the present disclosure provides a brain imaging system 100 includes a first imaging device 110, a second imaging device 120, a processor 130, and a memory 140. The processor 130 is electrically connected to the first imaging device 110, the second imaging device 120 and the memory 140.

The first imaging device 110 can be, for example, a computed tomography (CT) imaging device, which is configured to capture a plurality of first brain images by scanning a patient. The first brain images provide cerebral data representing a contrast agent in a brain of the patient over time, and the first brain images can be, for example, CT brain images. In some embodiments, the contrast agent can be, for example, an iodinated contrast agent. It should be noted that the brain images mentioned in the present disclosure generally refer to images captured with a field of view that covers the head and the neck of the patient.

Specifically, the second imaging device 120 can be, for example, a magnetic resonance imaging (MRI) device, which can be configured to capture a plurality of second brain images by scanning the patient. In some embodiments, the second brain images provide cerebral data representing another contrast agent in the brain of the patient over time, and the second brain images can be, for example, MRI brain images. In some embodiments, the contrast agent used for the second imaging device 120 can be, for example, a Gadolinium contrast agent. It should be noted that, the Gadolinium contrast agent can be, for example, Gadolinium-Diethylene Triamine Penta-acetic Acid (Gd-DTPA). Since Gd³⁺ in the lanthanide series is toxic and may lead to renal fibrosis as the excessive Gd³⁺ accumulates in human bodies, the Gd³⁺ is chelated by DTPA to form a stable compound, the Gd-DTPA.

However, in other embodiments, the second imaging device 120 is configured to capture the second brain images without using the contrast agent, and thus in such cases, the second brain images provide other cerebral data except for the contrast agent in the brain of the patient over time.

The processor 130 is configured to receive the first brain images and the second brain images from the first imaging device 110 and the second imaging device 120, convert the formats of the first brain images and the second brain images, and identify and analyze specific features in the converted first brain images and the converted second brain images, so as to generate cerebral perfusion data and lesion brain identification data of the patient.

More specifically, the processor 130 is configured to receive the first brain images and the second brain images from the first imaging device and the second imaging device, and distinguish the received brain images as the first brain images (e.g., CT images) or the second brain images (e.g., MRI images). In some embodiments, the first brain images and the second brain images are in DICOM (Digital Imaging and Communications in Medicine) format. Each of the first brain images includes a first mark, i.e., a first DICOM mark, and each of the second brain images includes a second mark, i.e., a second DICOM mark, different from the first mark. By the first mark and the second mark, the processor 130 is capable of distinguish the received brain images is the first brain images (e.g., CT images) or the second brain images (e.g., MRI images).

The processor 130 is further configured to convert the format of the first brain images into a first format, and convert the format of the second brain images into a second format. In some embodiments, the first format is nii format (Neuroimaging Informatics Technology Initiative file), and the second format is JPG/JPEG format. In other words, the processor 130 is configured to convert the first brain images into nii format, and convert the second brain images into JPG/JPEG format.

The processor 130 is further configured to identify first features in the converted first brain images through a first deep learning model, and identify second features in the converted second brain images through a second deep learning model. In some embodiments, the first features include cerebral blood flow (CBF) and maximum time (Tmax) when the contrast agent reaches a maximum calculate from the signals of the contrast agent. More specifically, in some embodiments, the first deep learning model includes long-short-term memory (LSTM) neural network to capture the best signal of the contrast agent in the blood flow, and calculate the CBF and Tmax, from which the positions of the ischemic core and the penumbra in the brain can be obtained.

As stated above, the identification of the first brain images (e.g., CT images) depends on the signal of the contrast agent in the cerebral blood flow, and the Singular Value Decomposition (SVD) is used for the calculation of such signal. In the present disclosure, the SVD model is used in the CT perfusion images for calculating the residual contrast agent function R(t) captured by the LSTM network. The relationship between the concentration of the contrast agent C(t) and the residual contrast agent function R(t) is shown in the following equation:

$$C ( t ) = {F}_{t} \cdot ( {C}_{a} ( t ) ⊗ R ( t ) ) .$$

wherein Cₐ(t) represents arterial input function (AIF), and Fₜ or CBF represents cerebral blood flow (CBF).

Therefore, the CBF of the contrast agent in the blood flow can be calculated by the following equation:

$${R}^{′} ( t ) = CBF R ( t ) .$$

On the other hand, to calculate the Tmax (the time when the concentration of the contrast agent reaches the maximum), the flow proportion residual contrast agent function k(t) should be considered, which is shown in the following equation:

$$k ( t ) = CBF {ρ}_{v o i} \cdot r ( t ) .$$

wherein the term “voi” represents volume of interest, and r(t) represents the residual contrast agent function.

Therefore, the Tmax can be presented as:

$$T max = {arg}_{t} max ( k ( t ) ) .$$

The physical meaning of this equation is that when the flow proportion residual contrast agent function reaches its maximum, the corresponding time is the Tmax when the concentration of the contrast agent reaches the maximum.

LSTM neural network is a specific type of recurrent neural network (RNN), and RNN is a network configured to process sequential data, such as image data having a time series. The interpretation of this type of data may be different because of the change of the environmental conditions with time, while RNN is capable of solve this kind of problem. Particularly, compared with common RNN, LSTM neural network has better performance in longer time series. Therefore, in the present disclosure, LSTM neural network is used for capturing the dynamic signal of the contrast agent in the first brain images (e.g., CT images), and calculating the flow speed of the contrast agent and Tmax by the SVD model.

For example, in the present embodiment, since perfusion data provided by the first brain image is sequential or temporal, the LSTM neural network can be a RNN with an LSTM architecture, which is trained to filter usable features for estimating brain perfusion indices. For example, the first brain images can be CT perfusion sequential images, which are included in each sample input vector jointly with patient-specific information and a value or values for one or more injection protocol parameters. The ground truth provided for each sample in training data include a perfusion parametric image, color-map image, quantitative values such as peak value, time to peak, cerebral blood flow (CBF), and/or cerebral blood volume (CBV), and/or cardiologist or radiologist decision (e.g., diagnosis and/or therapy), but the present disclosure is not limited thereto.

Specifically, the LSTM neural network is a type of RNN capable of learning order dependence in sequence prediction problems. As a consequence, it is also largely used to fit time-series data. An LSTM has a chain structure that includes four neural networks and several memory units known as cells. First, significant information is added to the neuron via the input gate. The forget gate then removes information that is no longer helpful in the present neuron state. The bias is applied to the current and prior inputs after they have been multiplied by the weight matrices. The result is fed into a binary activation function (similar to sigmoid). If the output state is zero, the information is deleted. If the output state is one, the information is saved for later use. Finally, the output gate is in charge of retrieving useful data from the neuron and sending it to the next neuron.

In some embodiments, the second deep learning model includes you-look-only-once (YOLO) deep learning model and region-convolutional neural network (R-CNN), by both of which the second features in the converted second images are identified. In some embodiments, the second features include a fluid-attenuated inversion recovery (FLAIR) and lightness increasing portions in diffusion-weighted images (DWI) of the converted second brain images.

The identification of the second brain images depends on the portions where the lightness of the pixels in the image increases. To achieve the detection of the lightness increasing portion(s) in the converted second brain images, YOLO deep learning model and R-CNN are used in the present disclosure. The YOLO deep learning model is a model used for object detection and supporting the training using custom dataset. In addition to the object detection tasks, YOLO deep learning model can also be used in object division. Practically, the whole image is used as the input to the YOLO deep learning model, and the input image is divided into plural parts. The coordinates of a bounding box that encloses the lightness increasing portion(s) is directly predicted. The bounding box of a target object includes the confidence score for the object detection and the evaluation of the category of the object. In this way, the result whether the bounding box encloses the lightness increasing portion(s) is/are identified based on the confidence score. Because of the architect and working principal described above, the YOLO network is more efficient in such type of calculation than other deep learning models, and can perform real-time detection in the image or the video and can detect plural target objects.

The R-CNN is also a tool used in object detection, but by a working principal different from the YOLO deep learning model. In the R-CNN, a selective search for the target object is performed to select thousands of proposal regions from the image or the video. Each of the proposal regions has a bounding box corresponding to the target object, and each of the proposal regions will be adjusted to a set size proportion (e.g., 277 pixels*277 pixels). The calculation steps of the R-CNN can be summarized as: (i) generating the proposal regions by the selective search; (ii) using a deep CNN to extract the features of the target object from each of the proposal regions; (iii) inputting the extracted features into the classifier of each category, and determining whether the features belong to the category; and (iv) using a bounding-box regression to modify the position of the bounding box. In this way, by setting the target object as the lightness increasing portion in the second brain images, the second features for identifying the low-blood-flow region in the brain can be captured and analyzed in a more objective manner than the naked-eye observations to the images.

In some embodiments, the processor 130 can include one or more processing units, and can be, for example, a central processing unit (CPU) and/or a general-purpose microprocessor, a microcontroller, a digital signal processor (DSP), a field programmable gate array (FPGA), a programmable logic device (PLD), and a combination of any of the above devices that can perform data calculation or other operations, or any other suitable circuits, devices and/or structures.

It should be noted that the processor 130 is electrically connected to a memory 140, and the memory 140 can be, for example, but not limited to, a hard disk, a solid-state disk, or other storage devices that can store data, which is configured to store at least a plurality of computer-readable instructions, data collections for training, the first brain images and the second brain images mentioned above. In some embodiments, the processor 130 and the memory 140 can be included in a computing device, such as a general-purpose computer is one that, given the application and required time, should be able to perform the most common computing tasks. Desktops, notebooks, smartphones and tablets, are all examples of general-purpose computers.

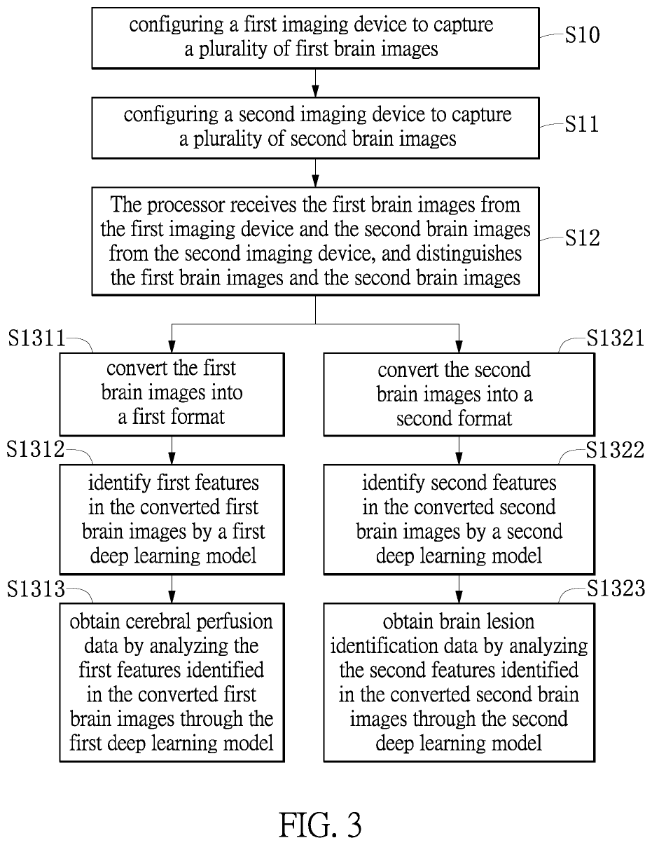

Reference is further made to FIG. 3, which shows a flowchart of a brain imaging method according to one embodiment of the present disclosure.

As shown in FIG. 3, the brain imaging method includes the following steps:

Step S10: configuring a first imaging device to capture a plurality of first brain images. In some embodiments, the first brain images can be CT brain images.

Step S11: configuring the second imaging device to capture the second brain images. In some embodiments, the second brain images can be MRI brain images.

The processor 130 is configured to perform the following steps:

Step S12: receiving the first brain images from the first imaging device and the second brain images from the second imaging device, and distinguishing the first brain images and the second brain images.

Based on the type of the received images, step S1311 to step 1313 or step 1321 to step 1323 are performed. If the received images are the first brain images, step S1311 to step 1313 will be taken. On the other hand, if the received images are the second brain images, step 1321 to step 1323 will be taken.

Step S1311: converting the first brain images into a first format. In some embodiments, the first format is a nii format.

Step S1312: obtaining and identifying first features in the converted first brain images through a first deep learning model. In some embodiments, the first features include CBF and Tmax when the contrast agent reaches a maximum calculated from the signals of the contrast agent, and the first deep learning model includes LSTM neural network for obtaining and identifying the first features in the converted first brain images. Therefore, the first features, including one or more of a cerebral blood flow (CBF), a cerebral blood volume (CBV), a cerebral blood mean transit time (MTT) and a first contrast agent time to peak (TTP) can be calculated and obtained according to the concentration curve, but the present disclosure is not limited thereto.

In particular, a vessel occlusion, infarction or ischemia region of the first brain images can be detected according to one of the cerebral blood flows, the cerebral blood volume, the cerebral blood mean transit time and the first contrast agent time to peak. Specifically, when the cerebral blood flow is below 30% of a normal cerebral blood flow, the cerebral blood volume is smaller than 40% of a normal cerebral blood volume and the first contrast agent time to peak is increasing, the processor 130, through the first imaging device 110, detects an infarct core of the vessel occlusion, infarction or ischemia region in the first brain image. In addition, when the cerebral blood flow is decreasing, the cerebral blood volume is maintained or increased, and the first contrast agent time to peak is dramatically increasing, the processor 130 through the first imaging device 110 detects a penumbra of the vessel occlusion, infarction or ischemia region in the first brain image.

Step S1313: obtaining cerebral perfusion data by analyzing the first features identified in the converted first brain images through the first deep learning model. In some embodiments, the cerebral perfusion data includes the positions of ischemic core and penumbra.

Step S1321: converting the second brain images into a second format. In some embodiments, the second format is a JPG/JPEG format.

Step S1322: identifying second features in the converted second brain images by a second deep learning model. In some embodiments, the second features include a fluid-attenuated inversion recovery (FLAIR), lightness increasing portions in diffusion-weighted images (DWI) of the converted second brain images and apparent diffusion coefficient (ADC) map of the DWIs, and the second deep learning model includes YOLO algorithm and R-CNN.

Step S1323: obtaining brain lesion identification data by analyzing the second features identified in the converted second brain images through the second deep learning model. In some embodiments, the brain lesion identification data includes a mismatch between the DWI and the FLAIR.

In order to perform data capturing and analysis with higher accuracy, the deep learning models can be trained for identifying different types of brain lesions. The deep learning models can include, for example, models such as LSTM neural network, YOLO and R-CNN used in the present disclosure. When identifying and calculating a volume of brain lesions, object detection and object recognition needs to be performed at once or in sequence, that is, one-stage or two-stage manner.

Also, to have better identification results and accuracy, the identification of each type of brain lesions is trained separately since the brain lesions may be too similar to one another. Therefore, the candidate deep learning models are trained by training sets having different types of images, respectively.

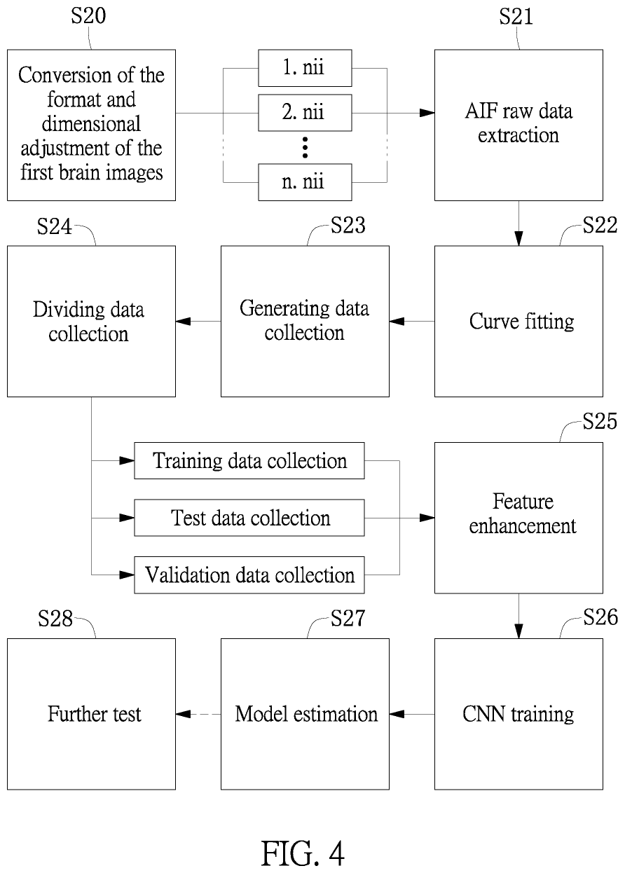

Reference can be made to FIG. 4, which shows a flowchart of training the first deep learning model mentioned above. The first deep learning model is trained through the following steps:

Step S20: converting the format of the first brain images and perform dimensional adjustment to the converted first brain images. In some embodiments, each of the first brain images is converted to a nii format, and forms a plurality of nii files shown in FIG. 4.

In some embodiments, before step S20, the first brain images can undergo an image pre-process for standardizing the input images. For example, a binarization method can be utilized, in which gray matter and white matter of the brain in the first brain images can be manually or automatically selected with a mask, and structures such as braincase and ventricles can be excluded For structures to be excluded by masking, pixel color values are 0 after the binarization, and for the target region to be retained, pixel color values are 1 multiply by original pixel values.

Specifically, the segmentation is an important stage of the image recognition system, because it extracts the objects of interest, for further processing such as description or recognition. Segmentation techniques are used to isolate the target region from the brain images in order to perform analysis.

As for the second brain images, because the second deep learning model will automatically identify the portions where the lightness increases and excludes the skull and the ventricle structures, the pre-process such as the standardization and the image division are optional for the second brain images.

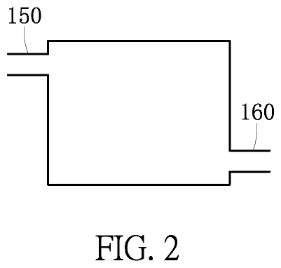

Step S21: capturing arterial input function (AIF) raw data from the converted first brain images as a source of the first features. In some embodiments, the AIF raw data includes an accumulated concentration function of the contrast agent and a residual concentration function of the contrast agent over time. FIG. 2 shows a simple model representing the contrast agent flowing in the brain of the patient is provided, which is related to the accumulated concentration function and the residual concentration function. In FIG. 2, positions of an entrance 150 and an exit 160 where the contrast agent flows into and out from the brain can identified from the first brain image, such that the concentration curve (i.e., a concentration curve of an iodinated contrast agent) can be obtained. In this embodiment, the concentration curve is a concentration curve of an iodinated contrast agent, and the contrast agent time to peak is an iodinated contrast agent time to peak. The processor 130 detects the position of the entrance 150 of the brain according to an iodinated contrast agents starting time, an iodinated contrast agents time to half-peak and the iodinated contrast agent time to peak.

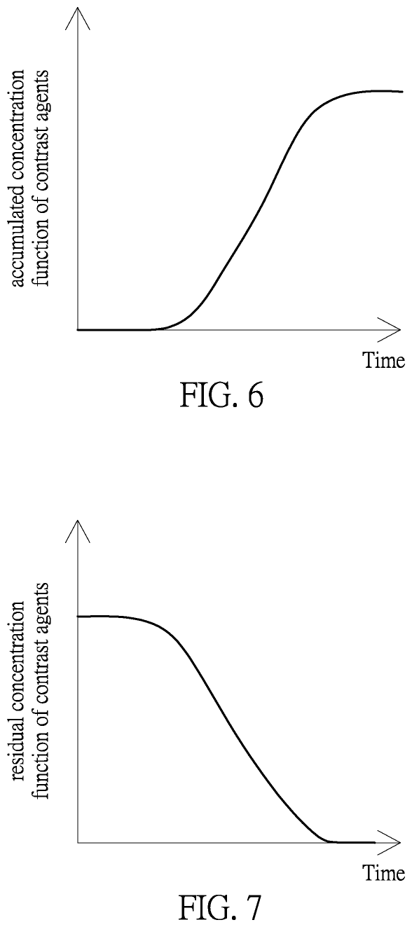

Reference can be made to FIG. 6 and FIG. 7, which respectively shows the accumulated concentration function and the residual concentration function of the contrast agent. FIGS. 6 and 7 show that, the accumulated concentration function of the contrast agent increases but the residual concentration function of the contrast agent decreases with time.

Step S22: fitting the curves of the AIF raw data. In some embodiments, the processor 130 is configured to calculate the cerebral blood flow, the cerebral blood volume, the cerebral blood mean transit time and the contrast agent time to peak according to the concentration curve showing the concentrations at the positions of the entrance 150 and the exit 160. Also, the processor 130 is further configured to detect the position of the entrance 150 iodinated according to the iodinated contrast agent starting time, the iodinated contrast agent time to half-peak and the iodinated contrast agent time to peak.

Step S23: generating a data collection including the converted first brain images.

Step S24: dividing the data collection into a training data collection, a test data collection and a validation data collection. In detail, the images in the training data collection can be the brain atlases pre-stored or pre-collected before training. The brain atlases can be made from multiple modalities and individuals provide the capability to describe image data with statistical and visual power. The brain atlases have enabled a tremendous increase in the number of investigations focusing on the structural and functional organization of the brain. In humans and other species, the brain's complexity and variability across subjects is so great that reliance on atlases is essential to manipulate, analyze and interpret brain data effectively.

The reference brain atlases can include, for example, initially intended to catalog morphological descriptions, brain atlases based upon 3D tomographic images, anatomic specimens and a variety of histologic preparations that reveal regional cytoarchitecture, brain atlases that include regional molecular content such as myelination patterns, receptor binding sites, protein densities and mRNA distributions, and other brain atlases describe function, quantified by positron emission tomography, functional MRI or electrophysiology, and the target brain atlas can be selected from above examples of the reference brain atlases.

Step S25: enhancing signals of the first features in the training data collection, the test data collection and the validation data collection.

Step S26: using a convolutional neural network (CNN) model to train the first deep learning model to identify the first features in the training data collection.

Step S27: estimating an identification result generated by the first deep learning model with the validation data collection.

Step S28: if the identification result generated by first deep learning model is validated, the first deep learning model is further tested by identifying the first features in the test data collection. If the identification result for the test data collection is also validated, the first deep learning model is determined to be usable for practical use.

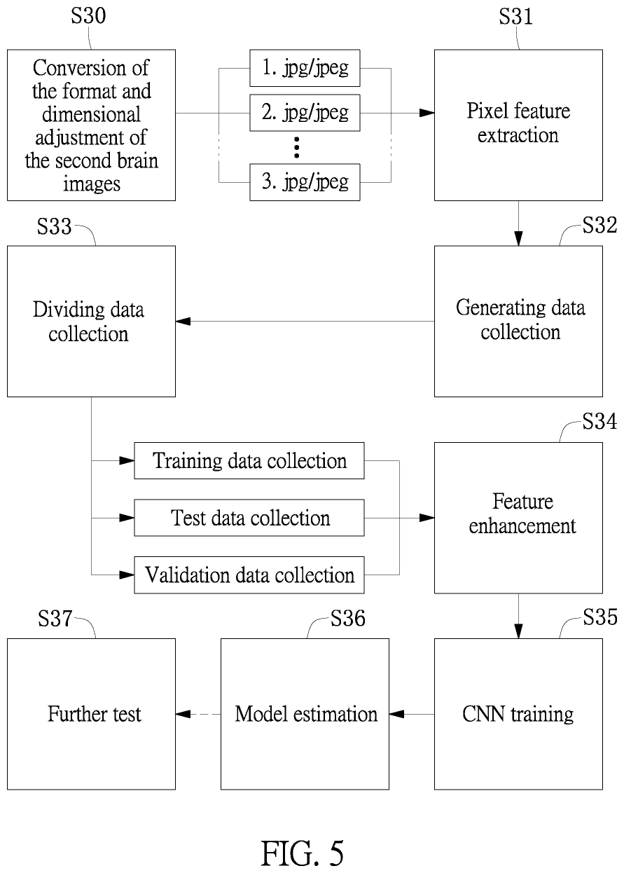

Reference can be made to FIG. 5, which shows a flowchart of training the second deep learning model mentioned above. As shown, the second deep learning model is trained through the following steps:

Step S30: converting the format of the second brain images and perform dimensional adjustment to the converted second brain images. In some embodiments, each of the second brain images is converted to a JPG/JPEG format, and forms a plurality of JPG/JPEG files shown in FIG. 5.

Step S31: extracting the second features from the converted second brain images. In some embodiments, the second features include a fluid-attenuated inversion recovery (FLAIR), lightness increasing portions in diffusion-weighted images (DWI) of the converted second brain images and apparent diffusion coefficient (ADC) map of the DWIs.

Step S32: generating a data collection including the converted second brain images.

Step S33: dividing the data collection into a training data collection, a test data collection and a validation data collection.

Step S34: enhancing signals of the second features in the training data collection, the test data collection and the validation data collection.

Step S35: using a convolutional neural network (CNN) model to train the second deep learning model to identify the second features in the training data collection.

Step S36: estimating an identification result generated by the second deep learning model with the validation data collection.

Step S37: if the identification result generated by second deep learning model is validated, the second deep learning model is further tested by identifying the second features in the test data collection. If the identification result for the test data collection is also validated, the second deep learning model is determined to be usable for practical use.

The YOLO (You Only Look Once) model used in the second deep learning model is a single-stage object detection algorithm that predicts the bounding boxes and class probabilities of objects in a single forward pass through the neural network. The YOLO algorithm at least includes a step of dividing an input brain image into a grid of cells, in which each cell is responsible for predicting a fixed number of bounding boxes and their associated class probabilities and each bounding box prediction consists of a set of values including x, y, width, height, and confidence score. Furthermore, non-maximum suppression is used to eliminate redundant bounding box predictions. The YOLO model consists of a convolutional neural network (CNN) that extracts features from the input image, followed by several fully connected layers that make the final predictions.

The R-CNN model is a two-stage object detection algorithm that first generates region proposals before predicting class probabilities and refining bounding boxes. The R-CNN algorithm at least includes steps of passing an input brain image through a CNN to extract feature maps, generating proposal regions that may contain objects by using a region proposal network (RPN), pooling and feeding the candidate regions into a classifier to predict the class probabilities and refine the bounding boxes, and eliminating redundant bounding box predictions by using non-maximum suppression.

In particular, the R-CNN model has two parts, i.e., a CNN that extracts features from the input image and an RPN that generates proposal regions for further processing. The RPN is trained to distinguish between foreground and background regions and to generate high-quality region proposals. The classifier is trained to classify the regions and refine the bounding boxes.

Assuming that there are 30 to-be-tested brain images, which are input to candidate deep learning models trained with DWIs and ADC images to generate detection results, such as DWI-1 and ADC-1, DWI-2 and ADC-2 . . . , DWI-30 and ADC-30, in which the target brain lesion is detected. The corresponding two detection results, such as DWI-1 and ADC-1, are compared to determine whether or not the detected target brain lesions in the two detection results having the same volumes and at the same positions, thereby determining whether or not the candidate deep learning model can be used to identify the one or more target brain lesions.

Furthermore, parameters of the candidate deep learning models having been trained can also be used to determine whether or not the candidate deep learning model can be used to identify the one or more target brain lesions. The parameters can include, for example, precision, recall, mean average precision (mAP) and other metric used to evaluate object detection models. In some embodiments, the one or more target brain lesions can include one or more of infarction areas, tumors, tumor metastasis, lymph nodes and lesions associated with dementia.

In addition to using the machine learned model, in the present disclosure, the infarct core of the vessel occlusion, infarction or ischemia region in the second brain image set can be detected by the processor 130 when the ADC is smaller than a diffusion threshold, or a penumbra of the vessel occlusion, infarction or ischemia region in the second brain image set can be detected by the processor 130 when the second contrast agent time to peak is larger than a time to peak. In practice, the ADC should be divided by 1,000,000, and the position of the brain image (x, y) includes two algebras referring to the position of the brain image. For example, the diffusion threshold can be 600 mm²/s, and the time to peak can be 6 seconds. The diffusion threshold and the time to peak can be further calculated by the processor 130 based on Bayesian statistics, but values are not limited in the present disclosure.

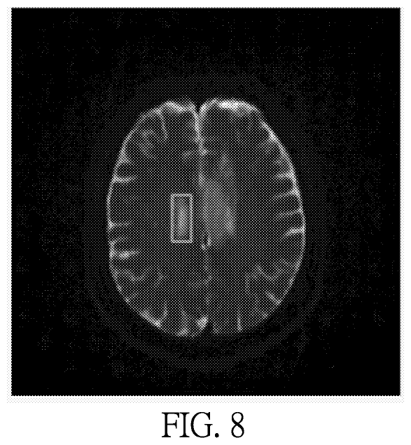

Reference is made to FIG. 8, which shows a schematic diagram of brain image according to one embodiment of the present disclosure. The processor 130 can execute an FMRIB Software Library (FSL) software to perform a Brain Extraction Tool to capture a calvarium image of the second brain image and separate the calvarium image from the second brain image. Then, the processor 130 divides the second brain image without the calvarium image into a plurality of brain regions. The processor 130 detects a penumbra of the vessel occlusion, infarction or ischemia region in the second brain image based on the Bayesian statistics. Specifically, according to a FSL instruction, the processor 130 divides the second brain image without the calvarium image into 15 brain regions, wherein these 15 brain regions include left brain regions and right brain regions. When the processor 130 receives the FSL instruction, the processor 130 uses the FSL software to do calculations for the cortex division, positions of the brain regions and volumes of the brain regions. The diffusion thresholds of the brain regions are different. Also, the diffusion thresholds of the brain regions may be varied due to age, gender or brain diseases. The processor 130 can determines the diffusion thresholds of all brain regions based on a big data analysis (e.g., the Bayesian statistics) to detect the penumbra of the vessel occlusion, infarction or ischemia region in the second brain image. Therefore, by using the FSL software, the processor 130 can not only divide the calvarium image from the second brain image, but also can calculate the volume of each brain region.

The processor 130 uses algorithms to generate an image of the vessel occlusion, infarction or ischemia region according to the vessel occlusion, infarction or ischemia region in the first brain image and the vessel occlusion, infarction or ischemia region in the second brain image. Specifically, the first brain image is the CT brain image, and the second brain image is the MRI brain image. Although the CT brain image has a low resolution with respect to the substantia nigra and the substantia alba, the CT brain image costs less time to be measured, being able to rapidly detect the vessel occlusion, infarction or ischemia region. On the other hand, the MRI brain image costs more time to be measured although the MRI brain image has a high resolution with respect to the substantia nigra and the substantia alba, which helps to find the long-term vessel occlusion, infarction or ischemia region and pathological changes around the vessel occlusion, infarction or ischemia region. In short, the CT brain image and the MRI brain image help detect the vessel occlusion, infarction or ischemia region, but both have pros and cons. Therefore, the present disclosure uses the algorithms to generate the image of the vessel occlusion, infarction or ischemia region according to the vessel occlusion, infarction or ischemia region detected by the CT and the MRI. Additionally, the present disclosure uses a set-up application to automatically examine whether there is a vessel occlusion, infarction or ischemia in a patient's brain, so that the medical staff would not need to determine whether there is a vessel occlusion, infarction or ischemia in a patient's brain by observing the brain image.

In conclusion, in the present disclosure, the CT brain images and the MRI brain images are captured respectively by the first imaging device and the second imaging device. Then, the brain images are optionally pre-processed and enhanced. Moreover, the LSTM model is further utilized to capture and analyze the dynamic image features (e.g., the CBF and Tmax) in the CT brain images for identifying brain ischemic core and penumbra. Additionally, the YOLO neural network and the R-CNN are used for capturing the lightness increase in the MRI brain images, so as to obtain the positions of the ischemic portion in the brain image.

In another aspect of the brain imaging system and the brain imaging method provided by the present disclosure, the CT brain images are converted into the concentration curves of the contrast agent to calculate the cerebral blood flow, the cerebral blood volume, the cerebral blood mean transit time and the contrast agent time to peak. Based on these features, the processor detects the vessel occlusion, infarction or ischemia region in the CT brain image through the first deep learning model. On the other hand, the lightness increase in the MRI brain images are detected and analyzed by the second deep learning model. In this way, the images of regions with the vessel occlusion, infarction or ischemia and the brain atrophy region are generated by algorithms to improve the conventional way of determining the positions of the vessel occlusion, infarction or ischemia region and the brain atrophy region. Therefore, the present disclosure effectively improves the efficiency and the precision of the examination of the brain vessel occlusion and dementia.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A brain imaging system, comprising:
a first imaging device, configured to capture a plurality of first brain images that provide cerebral data representing a contrast agent in a brain of the patient over time by scanning a patient;
a second imaging device, configured to capture a plurality of second brain images by scanning the patient; and
a processor electrically connected to the first image device and the second image device, wherein the processor is configured to:
receive the first brain images from the first imaging device and the second brain images from the second imaging device, and distinguish the first brain images and the second brain images;
convert the first brain images into a first format;
identify first features in the converted first brain images by a first deep learning model;
obtain cerebral perfusion data by analyzing the first features identified in the converted first brain images through the first deep learning model;
convert the second brain images into a second format;
identify second features in the converted second brain images by a second deep learning model; and
obtain brain lesion identification data by analyzing the second features identified in the converted second brain images through the second deep learning model;
wherein the first deep learning model includes a long-short-term memory (LSTM) neural network, by which the first features in the converted first brain images are identified; and
wherein the second deep learning model includes you-look-only-once (YOLO) algorithm and region-convolutional neural network (R-CNN), by both of which the second features in the converted second images are identified.

2. The brain imaging system according to claim 1, wherein the first deep learning model has been trained through the steps of:
capturing arterial input function (AIF) raw data from the converted first brain images as a source of the first features;
fitting curves generated from the AIF raw data;
generating a data collection including the converted first brain images;
dividing the data collection into a training data collection, a test data collection and a validation data collection;
enhancing signals of the first features in the training data collection, the test data collection and the validation data collection;
using a convolutional neural network (CNN) to train the first deep learning model to identify the first features in the training data collection; and
estimating an identification result generated by the first deep learning model with the validation data collection.

3. The brain imaging system according to claim 2, wherein, if the identification result generated by first deep learning model is validated, the first deep learning model is further tested by identifying the first features in the test data collection.

4. The brain imaging system according to claim 1, wherein the second deep learning model has been trained through the steps of:
extracting the second features from the converted second brain images;
generating a data collection including the converted second brain images;
dividing the data collection into a training data collection, a test data collection and a validation data collection;
enhancing signals of the second features in the training data collection, the test data collection and the validation data collection;
using a convolutional neural network (CNN) model to train the second deep learning model to identify the second features in the training data collection; and
estimating an identification result generated by the second deep learning model with the validation collection.

5. The brain imaging system according to claim 4, wherein, if the identification result generated by second deep learning model is validated, the second deep learning model is further tested by identifying the second features in the test data collection.

6. The brain imaging system according to claim 1, wherein each of the first brain images includes a first mark, each of the second brain images includes a second mark different from the first mark, and the first brain images and the second brain images are distinguished through the first mark and the second mark.

7. The brain imaging system according to claim 1, wherein the first brain images and the second brain images are of DICOM format, the first format is a nii format, and the second format is a JPG/JPEG format.

8. The brain imaging system according to claim 1, wherein the first image device is a computed tomography (CT) image device, the second image device is a magnetic resonance imaging (MRI) device; and the first brain images are CT brain images, the second brain images are MRI brain images.

9. The brain imaging system according to claim 8, wherein the first features include cerebral blood flow (CBF) and maximum time (Tmax) when the contrast agent reaches a maximum calculate from the signals of the contrast agent, and the cerebral perfusion data includes positions of ischemic core and penumbra.

10. The brain imaging system according to claim 1, wherein the second features include a fluid-attenuated inversion recovery (FLAIR), lightness increasing portions in diffusion-weighted images (DWI) of the converted second brain images and apparent diffusion coefficient map of the DWIs, and the brain lesion identification data includes a mismatch between the DWI and the FLAIR.

11. A brain imaging method, comprising:
configuring a first imaging device to capture a plurality of first brain images that provide cerebral data representing a contrast agent by scanning a patient;
configuring a second imaging device to capture a plurality of second brain images by scanning a patient;
configuring a processor, which is electrically connected to the first imaging device and the second imaging device, to:
receive the first brain images from the first imaging device and the second brain images from the second imaging device, and distinguish the first brain images and the second brain images;
convert the first brain images into a first format;
identify first features in the converted first brain images by a first deep learning model;
obtain cerebral perfusion data by analyzing the first features identified in the converted first brain images through the first deep learning model;
convert the second brain images into a second format;
identify second features in the converted second brain images by a second deep learning model; and
obtain brain lesion identification data by analyzing the second features identified in the converted second brain images through the second deep learning model;
wherein the first deep learning model includes a long-short-term memory (LSTM) neural network, by which the first features in the converted first brain images are identified; and
wherein the second deep learning model includes you-look-only-once algorithm (YOLO) and region-convolutional neural network (R-CNN), by both of which the second features in the converted second images are identified.

12. The brain imaging method according to claim 11, wherein the first deep learning model has been trained through the steps of:
capturing arterial input function (AIF) raw data from the converted first brain images as a source of the first features;
fitting curves generated from the AIF raw data;
generating a data collection including the converted first brain images;
dividing the data collection into a training data collection, a test data collection and a validation data collection;
enhancing signals of the first features in the training data collection, the test data collection and the validation data collection;
using a convolutional neural network (CNN) to train the first deep learning model to identify the first features in the training data collection; and
estimating an identification result generated by the first deep learning model with the validation data collection.

13. The brain imaging method according to claim 12, wherein, if the identification result generated by first deep learning model is validated, the first deep learning model is further tested by identifying the first features in the test data collection.

14. The brain imaging method according to claim 11, wherein the second deep learning model has been trained through the steps of:
extracting the second features from the converted second brain images;
generating a data collection including the converted second brain images;
dividing the data collection into a training data collection, a test data collection and a validation data collection;
enhancing signals of the second features in the training data collection, the test data collection and the validation data collection;
using a convolutional neural network (CNN) model to train the second deep learning model to identify the second features in the training data collection; and
estimating an identification result generated by the second deep learning model with the validation collection.

15. The brain imaging method according to claim 14, wherein, if the identification result generated by second deep learning model is validated, the second deep learning model is further tested by identifying the second features in the test data collection.

16. The brain imaging method according to claim 11, wherein each of the first brain images includes a first mark, each of the second brain images includes a second mark different from the first mark, and the first brain images and the second brain images are distinguished through the first mark and the second mark.

17. The brain imaging method according to claim 11, wherein the first brain images and the second brain images are of DICOM format, first format is nii format, and the second format is JPEG format.

18. The brain imaging method according to claim 11, wherein the first image device is a computed tomography (CT) image device, the second image device is a magnetic resonance imaging (MRI) device; and the first brain images are CT brain images, the second brain images are MRI brain images.

19. The brain imaging method according to claim 11, wherein the first features include cerebral blood flow (CBF) and maximum time (Tmax) when the contrast agent reaches a maximum calculate from the signals of the contrast agent, and the cerebral perfusion data includes positions of ischemic core and penumbra.

20. The brain imaging system according to claim 11, wherein the second features include a fluid-attenuated inversion recovery (FLAIR) and lightness increasing portions in diffusion-weighted images (DWI) of the converted second brain images, and the brain lesion identification data includes a mismatch between the DWI and the FLAIR.
